(19) United States
(12) Patent Application Publication
(10) Pub. No.: US 2026/0294227 A1
(43) Pub. Date: Oct. 1, 2026

(54) ROBOTIC SURGICAL SYSTEMS INCLUDING CONVECTION COOLED ENDOSCOPE

(71) Applicant: Covidien LP, Mansfield, MA (US)

(72) Inventors: Michael A. Zemlok, Prospect, CT (US); Burt D. Ochs, Methuen, MA (US)

(21) Appl. No.: 19/477,962

(22) PCT Filed: Apr. 10, 2024
(86) PCT No.: PCT/IB2024/053510

§ 371 Date: Oct. 23, 2025

Related U.S. Application Data
(60) Provisional application No. 63/462,043, filed on Apr. 26, 2023.

(51) Int. Cl. A61B 1/12; A61B 1/00; A61B 1/015; A61B 34/30

(52) U.S. Cl. CPC A61B 1/128; A61B 1/00006; A61B 1/015; A61B 34/30; A61B 2034/301

(57) ABSTRACT

A surgical system (10) includes a pressurized and regulated gas and/or air source for pumping insufflation gas into a patient, and an endoscope coupled to the pressurized and regulated gas and/or air source. The endoscope includes a thermal component. The endoscope further includes a convection tube (214) that extends through the endoscope. The convection tube is disposed in communication with the pressurized and regulated gas and/or air source. The convection tube is positioned adjacent to the thermal component and configured to enable the insufflation gas to be pumped through the endoscope by the pressurized and regulated gas and/or air source.

### CROSS-REFERENCE TO RELATED APPLICATION

This application claims the benefit of U.S. Provisional Patent Application Ser. No. 63/462,043, filed Apr. 26, 2023, the entire content of which is incorporated herein by reference.

### TECHNICAL FIELD

This disclosure relates to robotic surgical systems and, more particularly, to systems and methods for cooling robotic endoscopes.

### BACKGROUND

Robotic surgical systems include control drive assemblies supporting surgical instruments used in laparoscopic and/or robotic surgery. These surgical instruments generally have a proximally located actuating mechanism that is operably coupled to a control drive unit of the control drive assembly for actuating distal end effectors of the surgical instruments. The control drive unit includes any number of motors operably associated with the actuating mechanisms of the surgical instruments. A clinician remotely controls these motors to enable the surgical instruments to robotically perform a surgical task within a body cavity of a patient, and often in remote locations within the body cavity that are not easily accessed without robotic surgical systems.

The surgical instruments of these robotic surgical systems can include endoscopes with miniaturized, high-powered lights, high-definition cameras, and associated electronics that generate high, localized temperatures. Managing such temperatures, particularly in the distal ends of the smaller, minimally invasive endoscopes can be challenging. Excessive temperatures can damage the lights, the cameras, and/or the electronics. Endoscope surface temperatures are also controlled by limits that are required by medical regulatory compliance organizations.

### SUMMARY

According to an aspect of this disclosure, a robotic surgical system includes a control drive unit, a pressurized and regulated gas and/or air source for pumping insufflation gas into a patient, and an endoscope coupled to the pressurized and regulated gas and/or air source. The endoscope is selectively attached to the control drive unit. The endoscope includes at least one thermal component. The endoscope further includes at least one convection tube extending through the endoscope. The at least one convection tube is disposed in communication with the pressurized and regulated gas and/or air source. The at least one convection tube is positioned adjacent to the at least one thermal component. The at least one convection tube is configured to enable the insufflation gas to be pumped through the endoscope by the pressurized and regulated gas and/or air source for cooling the at least one thermal component by convection and for facilitating insufflation of the patient.

In aspects, the insufflation gas may include at least one of carbon dioxide, air, or an inert gas. The endoscope may extend to an end effector on a distal end portion of the endoscope. The end effector may define at least one cooling passage therethrough that enables the insufflation gas to flow into or out of a distal end of the endoscope. The at least one cooling passage may be disposed in communication with the at least one convection tube. The distal end portion may further include at least one temperature sensor configured to determine a temperature of at least one of the at least one thermal component or the at least one convection tube.

In aspects, the robotic surgical system may further include a controller. The temperature sensor may be disposed in communication with the controller. The pressurized and regulated gas and/or air source may be connected to the controller whereby the controller selectively actives the pressurized and regulated gas and/or air source when the temperature sensor exceeds a predetermined threshold temperature. The robotic surgical system may further include a valve assembly coupled to the endoscope. The at least one convection tube may be disposed in communication with the valve assembly. The controller may be configured to selectively actuate the valve assembly to adjust an amount of the insufflation gas being pumped through the at least one convection tube. The pressurized and regulated gas and/or air source may be configured to apply positive pressure to the insufflation gas through the at least one convection tube. The robotic surgical system may further include a negative pressurized and regulated gas and/or air source operatively coupled to the endoscope. The negative pressurized and regulated gas and/or air source may be disposed in communication with the at least one convection tube and configured to apply negative pressure to the insufflation gas through the at least one convection tube.

According to one aspect, a surgical system includes a pressurized and regulated gas and/or air source for pumping insufflation gas into a patient and an endoscope coupled to the pressurized and regulated gas and/or air source. The endoscope includes at least one thermal component. The endoscope further includes at least one convection tube extending through the endoscope. The at least one convection tube is disposed in communication with the pressurized and regulated gas and/or air source. The at least one convection tube is positioned adjacent to the at least one thermal component. The at least one convection tube is configured to enable the insufflation gas to be pumped through the endoscope by the pressurized and regulated gas and/or air source.

In aspects, a controller is configured to selectively actuate a valve assembly to adjust an amount of the insufflation gas being pumped through the at least one convection tube.

According to yet another aspect, a robotic surgical system includes a pressurized and regulated gas and/or air source for pumping insufflation gas into a patient, an endoscope coupled to the pressurized and regulated gas and/or air source, a processor, and a memory. The endoscope includes at least one thermal component that is configured to generate heat and at least one temperature sensor configured to sense an amount of heat generated by the at least one thermal component. The endoscope further includes at least one convection tube extending through the endoscope. The at least one convection tube is disposed in communication with the pressurized and regulated gas and/or air source for receiving the insufflation gas through the endoscope. The memory has instructions stored thereon, which when executed by the processor, cause the robotic surgical system to: determine an amount of heat generated by the at least one thermal component; and pump a predetermined amount of the insufflation gas through the at least one convection tube to cool the at least one thermal component by convection when the at least one temperature sensor senses that a temperature of the at least one thermal component exceeds a predetermined threshold.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, explain the principles of this disclosure, wherein:

FIG. 1 is a perspective view of a robotic surgical system being used for a surgical procedure on a patient in accordance with the principles of this disclosure;

FIG. 2 is an enlarged, perspective view of an endoscope of the robotic surgical system of FIG. 1;

FIG. 3 is an enlarged, cross-sectional view of the indicated area of detail shown in FIG. 2 as taken along section line 3-3;

FIG. 4 is an end view of FIG. 3; and

FIG. 5 is an enlarged schematic view of a cross-section of a camera assembly of the endoscope of FIG. 2.

### DETAILED DESCRIPTION

Aspects of this disclosure are described in detail with reference to the drawings, in which like reference numerals designate identical or corresponding elements in each of the several views. As used herein, the term “distal” refers to that portion of structure closer to a patient, while the term “proximal” refers to that portion of structure, farther from the patient. As used herein, the term “clinician” refers to a doctor, nurse, or other care provider and may include support personnel and/or equipment operators.

In the following description, well-known functions or constructions are not described in detail to avoid obscuring the present disclosure in unnecessary detail.

Robotic surgical systems have been used in minimally invasive medical procedures. Such procedures may be referred to as what is commonly referred to as “Telesurgery.” These robotic surgical systems have one or more surgical instruments removably coupled thereto. Such surgical instruments include, for example, endoscopes, electrosurgical forceps, cutting instruments, staplers, graspers, electrocautery devices, or any other endoscopic or open surgical devices. Prior to or during use of the robotic surgical system, various surgical instruments can be selected and connected to the robotic surgical system for selectively operating end effectors of the connected surgical instruments.

Given that the miniaturized, high-powered lights, high-definition camera assemblies, and associated electronics in endoscopes can generate high, localized temperatures, this disclosure details structures and methods for cooling and controlling endoscope temperatures through convection cooling (e.g., air and/or gas such as carbon dioxide or inert gas). By enabling pressurized air and/or gases to flow through the endoscope from a pressurized source that is either external or internal to a patient, sufficient heat removal can be consistently obtained for optimal light emission, and high definition, 3D, digital video transmission. Advantageously, to effectuate convection cooling, the disclosed endoscopes utilize pressurized insufflation air and/or gas that is currently used within the patient for laparoscopic or robotic minimally invasive surgeries. By enabling pressurized air and/or gas within the patient to flow in through the distal end portion of the endoscope, and out through the endoscope external to the patient, sufficient convection is attained to cool the primary components of concern within the distal end portion of the endoscope.

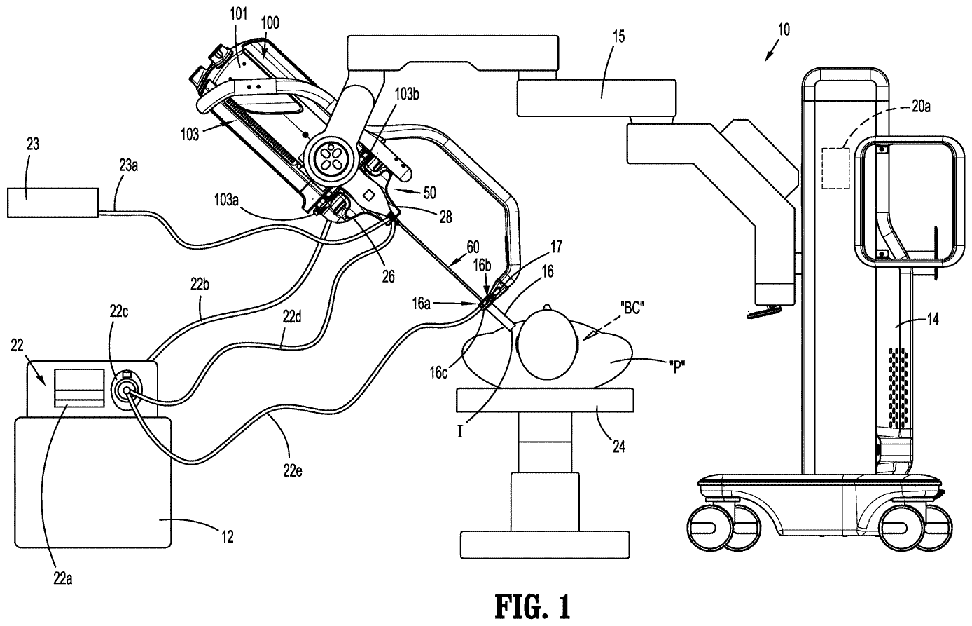

With reference to FIG. 1, a robotic surgical system is shown generally at 10. Robotic surgical system 10 employs various robotic elements to assist the clinician and allow remote operation (or partial remote operation) of surgical instruments 60 of surgical instrument systems 50 of robotic surgical system 10. Various controllers, circuitry, robotic arms, gears, cams, pulleys, electric and mechanical motors, etc. may be employed for this purpose and may be designed with surgical system 10 to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic surgical system 10 includes a workstation 12 and an instrument cart 14. Instrument cart 14 supports a movable control drive assembly 100 on a movable setup arm assembly 15 that are selectively movable relative to instrument cart 14. Instrument cart 14 supports and positions movable setup arm assembly 15 in proximity to the patient “P” within an operating theater. Control drive assembly 100 includes one or more surgical instrument systems 50 mounted on a control drive unit 101 supported on setup arm assembly 15. Control drive unit 101 is movable relative to cart 14 and houses an instrument drive assembly 103 for manipulating surgical instrument systems 50 and/or independent surgical instruments 60 thereof with the assistance of, for example, one or more computing devices or controllers. For instance, instrument drive assembly 103 can include one or more instrument drive units 103a for operating surgical devices such as graspers coupled thereto, and an endoscope drive unit 103b for operating surgical devices such as an endoscope 60a coupled thereto. Surgical instrument system 50 can include any number and/or type of surgical instruments. Surgical instruments 60 can include, for example, graspers or forceps 26, which may be electrosurgical, an endoscope 28, and/or any other suitable instrument that can be driven by one or more associated tool drives, such as instrument drive unit 103a and endoscope drive unit 103b of instrument drive assembly 103. For example, besides graspers 26 and endoscope 28, surgical instruments 60 can include dexterous tools, such as grippers, needle drivers, staplers, dissectors, cutters, hooks, graspers, scissors, coagulators, irrigators, suction devices, which are used for performing a surgical procedure.

Surgical instrument system 50 of robotic surgical system 10 further includes a surgical portal assembly 16 defining a plurality of separate and spaced-apart conduits, channels or lumens 16a, 16b therethrough that are configured to receive, for instance, surgical instruments 60 for accessing a body cavity “BC” (e.g., a peritoneal cavity) of a patient “P.” Such lumens 16a, 16 may be of any suitable shape and/or size. In other aspects, surgical portal assembly 16 may define a single conduit, channel, or lumen therethrough that is configured to receive, for instance, surgical instruments 60 for accessing the body cavity “BC” of the patient “P”. In particular, surgical portal assembly 16 can be inserted through an incision “I” and into the body cavity “BC” of the patient “P”. Surgical portal assembly 16 is coupled to control drive assembly 100 via a mounting member 17 that supports surgical portal assembly 16 relative to control drive unit 101 and surgical instruments 60.

Workstation 12 of robotic surgical system 10 includes an input device 22 in communication with control drive unit 101 for use by a clinician to control surgical portal assembly 16 and the various surgical instrument systems 50 (and surgical instruments 60 thereof) via instrument drive assembly 103 for performing surgical operations on the patient “P” while the patient “P” is supported on a surgical table 24, for example. Input device 22 is configured to receive input from the clinician and is configured to produce input signals. Input device 22 may also be configured to generate feedback to the clinician. The feedback can be visual, auditory, haptic, or the like.

Workstation 12 of robotic surgical system 10 can further include computing devices and/or controllers such as a master processor circuit 22a in communication with input device 22 for receiving the input signals and generating control signals for controlling robotic surgical system 10, which can be transmitted to instrument cart 14 via an interface cable 22b. In some cases, transmission can be wireless and interface cable 22b may not be present. Input device 22 can include right and left-hand controls (not shown) and/or foot pedals (not shown), which are moved/operated to produce input signals at input device 22 and/or to control robotic surgical system 10. Instrument cart 14 can include a slave processor circuit 20a that receives the control signals from master processor circuit 22a and produces slave control signals operable to control surgical instrument systems 50 (and surgical instruments 60 thereof) during a surgical procedure. Workstation 12 can also include a user interface, such as a display (not shown) in communication with the master processor circuit 22a for displaying information (such as, body cavity images) for a region or site of interest (for example, a surgical site, a body cavity, or the like) and other information to a clinician. While both master and slave processor circuits are illustrated, in other aspects, a single processor circuit may be used to perform both master and slave functions.

In aspects, input device 22 can include an insufflation and/or positive pressurized and regulated gas and/or air source 22c that introduces an inert gas and/or air at a positive pressure (or greater than ambient pressure) into the patient “P” via a first pressurized line 22d to provide additional access within the patient's body cavity “BC”. In aspects, pressurized and regulated gas and/or air source 22c may include a pressure pump. In particular, first pressurized line 22d is coupled to endoscope 28 (e.g., via a valve assembly 210) to pressurize endoscope 28—while endoscope 28 is in the patient—and to enable gas and/or airflow through endoscope 28 and into the patient “P” to provide endoscope convection cooling and insufflation to the patient “P”. A second pressurized line 22e extends between insufflation and/or positive pressurized and regulated gas and/or air source 22c and a valve 16c of surgical portal assembly 16 which can also be used for insufflating patient “P” in minimally invasive surgery. Pump 22c may be separate and distinct from input device 22.

The insufflation gas, which may include carbon dioxide, inert gases, and/or air, can be pumped into the body cavity “BC” of the patient “P”, such as the peritoneal cavity, to produce a state of pneumoperitoneum. This creates an increase in the intra-abdominal pressure (IAP). The peritoneal cavity can be insufflated at any suitable rate, for example, as high as about 40 liters/min or less (e.g., high flow systems) to a pressure as high as about 30 mm Hg or less. In some instances, the peritoneal cavity can be insufflated at a rate of 4-6 liters/min (e.g., low flow systems) to a pressure of about 10-20 mm Hg. The pneumoperitoneum may be maintained by a constant gas flow of up to about 40 liters/min, and in some instances, to a pressure of about 200-400 ml/min.

An external positive or negative pressure (e.g., vacuum) pump 23 of robotic surgical system 10 can be used to provide negative and/or positive pressure (e.g., air or inert gas) within endoscope 28 to provide a desired flow for adequate convection for air and/or inert gas cooling of endoscope 28 (or portions thereof). Pump 23 can be coupled to endoscope 28 via line 23a (e.g., via one or more valves of valve assembly 210 of endoscope 28). For example, negative pressurized and regulated gas and/or air source 23 can be used to pull air and/or gas through the endoscope (e.g., in a distal to proximal direction), and external to the patient, to achieve the desired airflow of up to about 6 liters/min based on flow restrictions in the endoscope 28.

Due to the small areas and restrictions for gas and airflow, the low pressures of insufflation pumps may not create an adequate flow rate for the desired convection cooling. Thus, it is envisioned that higher pressurized and regulated gas and/or air sources (e.g., greater than 20 mm Hg) can be used to generate the desired gas or airflow. The pump and/or vacuum flow methods can utilize any number of temperature sensors 216 for monitoring energy usage of the components within the distal end portion 200b of endoscope 28 for a control feedback loop to increase the pressure (e.g., positive and/or negative pressure) and/or vacuum required for the desired airflow needed for adequate convection cooling.

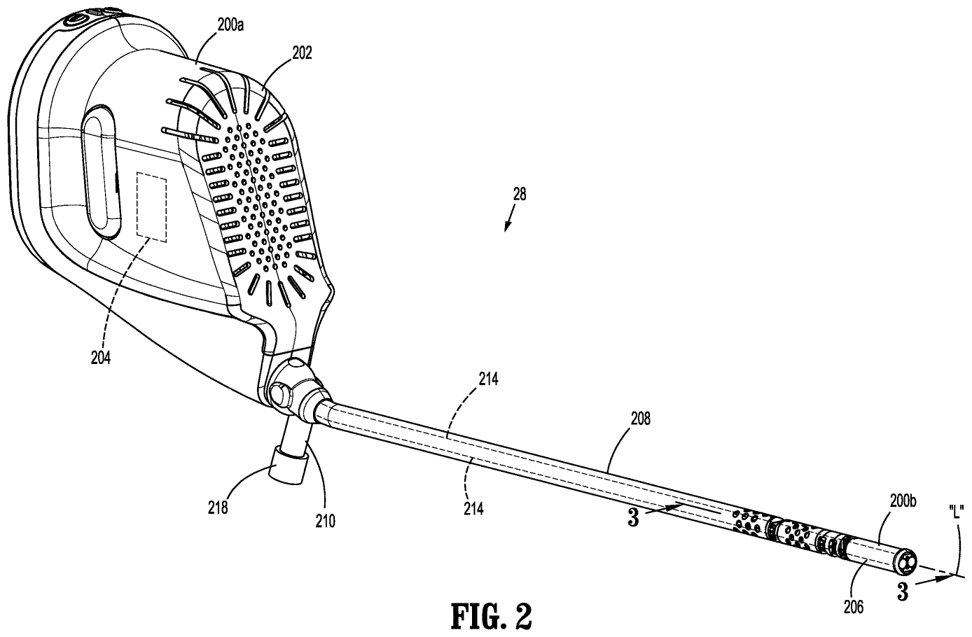

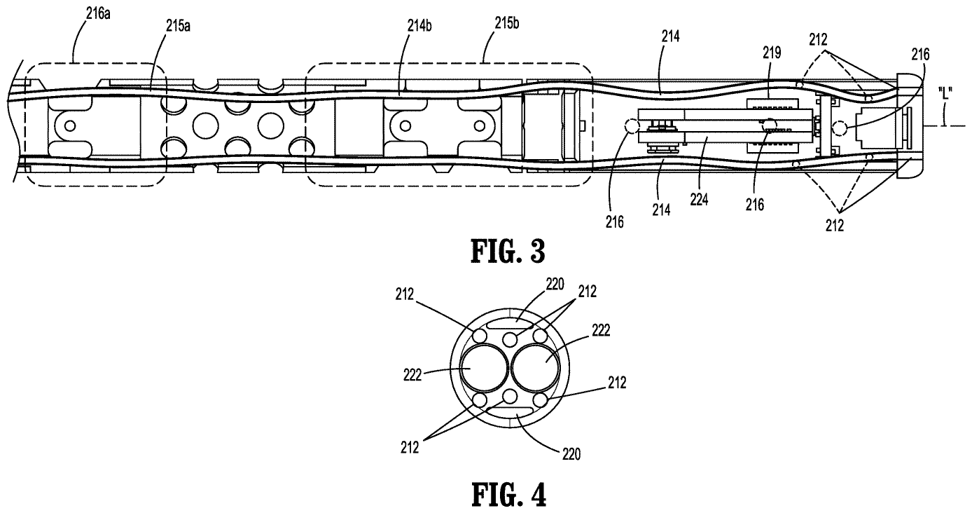

As seen in FIG. 2, endoscope 28 defines a longitudinal axis “L” and includes a proximal end portion 200a and a distal end portion 200b that supports various thermal components (e.g., for effectuating in vivo visualization), and which can generate heat, such as electronics 219, cameras 222, lights 220, and/or other power, communication, and/or sensing components 224 for the cameras 222, lights 220, and/or electronics 219, and which may require cooling and/or thermal monitoring. A housing 202 on the proximal end portion 200a of endoscope 28 supports a drive assembly 204 that is operatively coupled to an end effector 206 on the distal end portion 200b of endoscope 28 for operating and/or moving (e.g., articulating) end effector 206.

An elongated shaft assembly 208 of endoscope 28 connects housing 202 to end effector 206. Elongated shaft assembly 208 is sealed and supports power wires, communication wires, fiber optic light cables, and/or the like therein. Elongated shaft assembly 208 includes a valve assembly 210. Valve assembly 210 of endoscope 28, which may include any number of valves (which may be coupled together and/or discrete from one another), can be coupled to endoscope 28 at any suitable location such as at a proximal end portion of elongated shaft assembly 208. Valve assembly 210 may be an insufflation valve for minimally invasive patient pressurization where pressurized insufflation gas can be connected to and fed through endoscope 28 to conventionally cool the electronics, cameras, light components, etc., supported within the distal end portion 200b of endoscope 28.

Elongated shaft assembly 208 defines any number of cooling passages 212 of endoscope 28 that enable air/gas flow into and/or out of the distal end portion 200b of endoscope 28. These passages 212 have cross sections and restricted areas within the endoscope 28 to maximize airflow for convection cooling and to reduce blockage concerns. The one or more cooling passages 212 can be optimized to flow across and/or through and/or in close proximity to the most thermally vulnerable and/or thermal generating components of endoscope 28. Elongated shaft assembly 208 further supports a plurality of elongated hollow convection tubes 214 therein that are disposed in fluid communication with valve assembly 210. Tubes 214 (e.g., airlines) can be rigid and/or flexible. Tubes 214 and can be used through any bendable, articulating, and/or compliant sections or joints at any point on, or within, endoscope 28 and/or elongated shaft assembly 208 of endoscope 28. For instance, tubes 214 can include flexible sections 214a, 214b incorporated in flexible regions or joints 215a, 215b incorporated along any portion of the elongated shaft assembly 208, which may be in the form of compliant, bendable, pivoting, and/or articulating joints within the distal end portion 200b of elongated shaft assembly 208 to enable movement (e.g., multiple degrees of freedom) of the distal end portion 200a for improving access and positioning capabilities for the endoscope camera.

In aspects, valve assembly 210 may be a pneumatic bleed valve that may be fixed or variable to enable insufflation gases within the patient to be channeled through the one or more cooling passages 212 defined through distal end portion 200b and/or through the one or more hollow convection tubes 214 extending through endoscope 28 to conventionally cool the distal electronics and light components supported by the distal end portion 200b.

In aspects, endoscope 28 provides airflow through endoscope 28 having a static exhaust area and/or can be controlled by a “dynamic” variable area. Valve assembly 210, cooling passages 212, and/or convection tubes 214 provide a dynamic or variable exhaust system that can include adjustable exhaust orifices, valves (e.g., servo valve, variable exhaust valve), and/or Nitinol shape memory material for selectively enabling more and/or less air and/or gas flow (e.g., opening and/or closing) therethrough. For instance, valve assembly 210, convection tubes 214, and/or cooling passages 212 can be tuned to actuate after surpassing one or more predetermined temperature thresholds.

Valve assembly 210, convection tubes 214, and/or cooling passages 212 can be controlled (e.g., automatically) and/or adjusted based on temperature changes (e.g., Nitinol expansion and/or retraction temperature-based response) and/or from feedback that is monitored and obtained from the one or more temperature sensors 216 that are placed, for example, in proximity to a high temp light source and/or components within end effector 206 and/or within elongated shaft assembly 208 of endoscope 28. In particular, air and/or gas exhaust, which is external to the patient “P”, can be controlled by opening (or unrestricting) or closing (or restricting) valve assembly 210 (e.g., servo valve), convection tubes 214, and/or cooling passages 212 based on endoscope energy usage (e.g., momentarily or over a running and/or predetermined time period) or by a control feedback loop based on feedback data generated from the one or more temperature sensors 216 and communicated to a controller of the robotic surgical system 10 for selectively causing the valve assembly 210, for example, to adjust (open and/or close) as needed. For instance, a servo-valve assembly (not shown) can be incorporated in valve assembly 210 to control and increase the exhaust valve area for increasing the exhaust airflow for high energy lights, high energy cameras, and/or electronics disposed in the distal end portion 200b of the endoscope 28. This cooling feature can also be controlled through a feedback loop to temperature sensors 216 which are to open to increase flow if high temperature thresholds are met or surpassed. In particular, temperature sensors 216 are placed within proximity to thermal generating, thermally vulnerable, and/or thermally conductive materials and/or components within the end effector 206 of endoscope 28 for providing a control feedback loop that increases convection and/or cooling capabilities and/or thermal conditions and/or energy use conditions for endoscope 28.

Temperature sensors 216 of endoscope 28 can monitor internal component temperatures, external component temperatures, heat sink temperatures, conductive medium temperatures, elongated shaft assembly temperatures, and/or the distal end temperatures for a control feedback loop to increase airflow for cooling. Energy and/or current flow over time can also be monitored to control the thermal conditions and/or airflow in such a feedback loop. The temperature sensing and/or feedback control loop can also digitally drive a lower power mode for light, definition, and/or communication speed to reduce the heat generation after a predetermined temperature threshold is surpassed.

In some aspects, valve assembly 210 can include an air filter assembly 218 that filters air entering the endoscope 28 through valve assembly 210. N95 or finer filtering of any gas and/or air that is pumped into or exhausted from the patient by a positive pressurized and regulated gas and/or air source, a negative external pressure differential, from insufflation and/or through a negative pressure vacuum pump can be provided to prevent any viral and/or bacterial exposure to the patient and/or clinical workers in proximity of the device or patient.

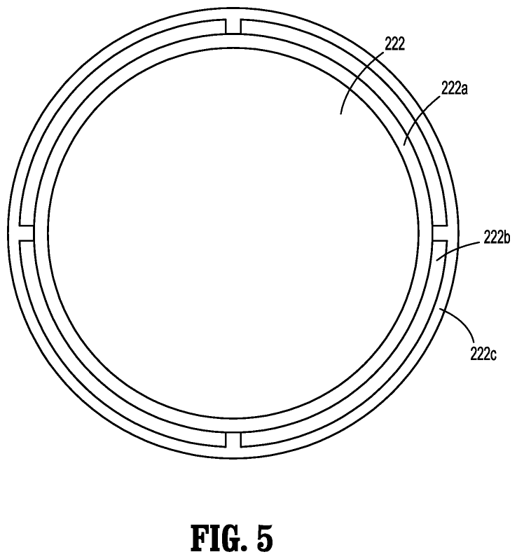

With reference to FIG. 5, portions of endoscope 28 can include different structures to help facilitate convection cooling. For instance, camera 222 can be surrounded by a thermally conductive material layer 222a, an annular air channel 222b about the thermally conductive material layer 222a that generates flow from insufflation, and/or a flexible thermally resistive material layer 222c that extends about annular air channel 222b.

Thermally conductive materials and/or heat sink components around or in proximity to, and in aspects, in contact with, the components generating heat (e.g., lights, digital cameras, power electronics, and/or communication electronics) or the heat sink components or materials, can be used to conduct heat away from the thermally generating components. Advantageously, the thermally conductive materials and/or heat sink components increase the thermal mass and the area around the thermally generating components to provide larger areas for cooling through convection.

The thermally conductive materials can be composed of any conductive metal alloy such as aluminum, copper, iron, gold, sliver, tin, and/or or lead alloys that may also include thermally conductive plating materials and/or coatings. It is also envisioned that the components and/or the entire distal end (or portions thereof) of endoscope 28, which includes the thermal generating components, may be potted and/or over-molded with thermally conductive materials. The materials and/or additives to the potting and/or over-mold can include thermally conductive and/or electrically resistive materials and compounds such as boron nitride, aluminum nitride, mica, carbon, ceramics and/or silicone. These materials can be in contact with the internal components and/or the external components of endoscope 28, such as components of elongated shaft assembly 208, or through the air and/or gas convection cooling methods within endoscope 28. The potting, over-molding, heatsink, and/or coatings are thermally conductive to provide a conductive heatsink mass or masses to thermal generating or thermally vulnerable components, and include geometries that enable airflow around or through them to provide either proximal to distal or distal to proximal convection airflow or gas cooling to the distal end of the endoscope.

The disclosed structure can include any suitable mechanical, electrical, and/or chemical components for operating the disclosed system or components thereof. For instance, such electrical components can include, for example, any suitable electrical and/or electromechanical, and/or electrochemical circuitry, which may include or be coupled to one or more printed circuit boards. As appreciated, the disclosed computing devices (and/or servers) can include, for example, a “controller,” “processor,” “digital processing device” and like terms, and which are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed apparatus and provides services for execution of applications for use with the disclosed apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tape drives, optical disk drives, and cloud-computing-based storage. In various aspects, the storage and/or memory device is a combination of devices such as those disclosed herein.

In various aspects, the memory can be random access memory, read-only memory, magnetic disk memory, solid state memory, optical disc memory, and/or another type of memory. In various aspects, the memory can be separate from the controller and can communicate with the processor through communication buses of a circuit board and/or through communication cables such as serial ATA cables or other types of cables. The memory includes computer-readable instructions that are executable by the processor to operate the controller. In various aspects, the controller may include a wireless network interface to communicate with other computers or a server. In aspects, a storage device may be used for storing data. In various aspects, the processor may be, for example, without limitation, a digital signal processor, a microprocessor, an ASIC, a graphics processing unit (“GPU”), field-programmable gate array (“FPGA”), or a central processing unit (“CPU”).

The memory stores suitable instructions and/or applications, to be executed by the processor, for receiving the sensed data (e.g., sensed data from camera), accessing storage device of the controller, generating a raw image based on the sensed data, comparing the raw image to a calibration data set, identifying an object based on the raw image compared to the calibration data set, transmitting object data to a post-processing unit, and displaying the object data to a graphic user interface. Although illustrated as part of the disclosed structure, it is also contemplated that a controller may be remote from the disclosed structure (e.g., on a remote server), and accessible by the disclosed structure via a wired or wireless connection. In aspects where the controller is remote, it is contemplated that the controller may be accessible by, and connected to, multiple structures and/or components of the disclosed system.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on the disclosed controllers or on a user device, including for example, on a mobile device, an IOT device, or a server system.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the disclosed apparatus, components thereof, and/or resources thereof. As used herein, the term “network” can include any network technology including, for instance, a cellular data network, a wired network, a fiber-optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed system or components thereof.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

The phrases “in an aspect,” “in aspects,” “in various aspects,” “in some aspects,” “in other aspects” or the like may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to all such states. Reference to a program may encompass the actual instructions and/or the intent of those instructions.

Securement of any of the components of the disclosed devices may be effectuated using known securement techniques such welding, crimping, gluing, fastening, etc.

The subject matter of this disclosure may be further described by reference to the following numbered aspects:

1. A robotic surgical system, comprising:
a control drive unit;
a pressurized and regulated gas and/or air source for pumping insufflation gas into a patient; and
an endoscope coupled to the pressurized and regulated gas and/or air source and selectively attached to the control drive unit, the endoscope including at least one thermal component, the endoscope further including at least one convection tube extending through the endoscope, the at least one convection tube disposed in communication with the pressurized and regulated gas and/or air source, the at least one convection tube positioned adjacent to the at least one thermal component, the at least one convection tube configured to enable the insufflation gas to be pumped through the endoscope by the pressurized and regulated gas and/or air source for cooling the at least one thermal component by convection and for facilitating insufflation of the patient.

2. The robotic surgical system of aspect 1, wherein the insufflation gas includes at least one of carbon dioxide, air, or an inert gas.

3. The robotic surgical system of aspect 2, wherein the endoscope extends to an end effector on a distal end portion of the endoscope.

4. The robotic surgical system of aspect 3, wherein the end effector defines at least one cooling passage therethrough that enables the insufflation gas to flow into or out of a distal end of the endoscope.

5. The robotic surgical system of aspect 4, wherein the at least one cooling passage is disposed in communication with the at least one convection tube.

6. The robotic surgical system of aspect 5, wherein the distal end portion further includes at least one temperature sensor configured to determine a temperature of at least one of the at least one thermal component or the at least one convection tube.

7. The robotic surgical system of aspect 6, further comprising a controller, wherein the temperature sensor is disposed in communication with the controller, and wherein the pressurized and regulated gas and/or air source is connected to the controller whereby the controller selectively actives the pressurized and regulated gas and/or air source when the temperature sensor exceeds a predetermined threshold temperature.

8. The robotic surgical system of aspect 7, further comprising a valve assembly coupled to the endoscope, the at least one convection tube disposed in communication with the valve assembly.

9. The robotic surgical system of aspect 8, wherein the controller is configured to selectively actuate the valve assembly to adjust an amount of the insufflation gas being pumped through the at least one convection tube.

10. The robotic surgical system of aspect 9, wherein the pressurized and regulated gas and/or air source is configured to apply positive pressure to the insufflation gas through the at least one convection tube, and further comprising a negative pressurized and regulated gas and/or air source operatively coupled to the endoscope, the negative pressurized and regulated gas and/or air source disposed in communication with the at least one convection tube and configured to apply negative pressure to the insufflation gas through the at least one convection tube.

11. A surgical system, comprising:
a pressurized and regulated gas and/or air source for pumping insufflation gas into a patient; and
an endoscope coupled to the pressurized and regulated gas and/or air source, the endoscope including at least one thermal component, the endoscope further including at least one convection tube extending through the endoscope, the at least one convection tube disposed in communication with the pressurized and regulated gas and/or air source, the at least one convection tube positioned adjacent to the at least one thermal component, the at least one convection tube configured to enable the insufflation gas to be pumped through the endoscope by the pressurized and regulated gas and/or air source.

12. The surgical system of aspect 11, wherein the insufflation gas includes at least one of carbon dioxide, air, or an inert gas.

13. The surgical system of aspect 12, wherein the endoscope extends to an end effector on a distal end portion of the endoscope, wherein the end effector defines at least one cooling passage therethrough that enables the insufflation gas to flow into or out of a distal end of the endoscope.

14. The surgical system of aspect 13, wherein the at least one cooling passage is disposed in communication with the at least one convection tube.

15. The surgical system of aspect 14, wherein the distal end portion further includes at least one temperature sensor configured to determine a temperature of at least one of the at least one thermal component or the at least one convection tube.

16. The surgical system of aspect 15, further comprising a controller, wherein the temperature sensor is disposed in communication with the controller, and wherein the pressurized and regulated gas and/or air source is connected to the controller whereby the controller selectively actives the pressurized and regulated gas and/or air source when the temperature sensor exceeds a predetermined threshold temperature.

17. The surgical system of aspect 16, further comprising a valve assembly coupled to the endoscope, the at least one convection tube disposed in communication with the valve assembly.

18. The surgical system of aspect 17, wherein the controller is configured to selectively actuate the valve assembly to adjust an amount of the insufflation gas being pumped through the at least one convection tube.

19. The surgical system of aspect 18, wherein the pressurized and regulated gas and/or air source is configured to apply positive pressure to the insufflation gas through the at least one convection tube, and further comprising a negative pressurized and regulated gas and/or air source operatively coupled to the endoscope, the negative pressurized and regulated gas and/or air source disposed in communication with the at least one convection tube and configured to apply negative pressure to the insufflation gas through the at least one convection tube.

20. A robotic surgical system, comprising:
a pressurized and regulated gas and/or air source for pumping insufflation gas into a patient;
an endoscope coupled to the pressurized and regulated gas and/or air source, the endoscope including at least one thermal component that is configured to generate heat and at least one temperature sensor configured to sense an amount of heat generated by the at least one thermal component, the endoscope further including at least one convection tube extending through the endoscope, the at least one convection tube disposed in communication with the pressurized and regulated gas and/or air source for receiving the insufflation gas through the endoscope;
a processor; and
a memory having instructions stored thereon, which when executed by the processor, cause the robotic surgical system to:
   determine an amount of heat generated by the at least one thermal component; and
   pump a predetermined amount of the insufflation gas through the at least one convection tube to cool the at least one thermal component by convection when the at least one temperature sensor senses that a temperature of the at least one thermal component exceeds a predetermined threshold.

21. A method for cooling a robotic surgical system, the method comprising:
determining an amount of heat generated by at least one thermal component of an endoscope; and
pumping a predetermined amount of insufflation gas through at least one convection tube extending through the endoscope when at least one temperature sensor of the endoscope senses that a temperature of the at least one thermal component exceeds a predetermined threshold.

22. The method according to aspect 21, further comprising executing instructions stored on a memory with a processor of the robotic surgical system, wherein the instructions cause a pump of the robotic surgical system to pump the predetermined amount of insufflation gas based on a determined amount of heat generated by the at least one thermal component.

23. The method according to aspect 21 or aspect 22, further comprising cooling the at least one thermal component as the insufflation gas passes through the at least one convection tube.

24. The method according to aspect 23, wherein cooling the at least one thermal component is effectuated by convection.

Persons skilled in the art will understand that the structures and methods specifically described herein and shown in the accompanying figures are non-limiting exemplary aspects, and that the description, disclosure, and figures should be construed merely as exemplary of aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

1. A robotic surgical system, comprising:
a control drive unit;
a pressurized and regulated gas and/or air source for pumping insufflation gas into a patient; and
an endoscope coupled to the pressurized and regulated gas and/or air source and selectively attached to the control drive unit, the endoscope including at least one thermal component, the endoscope further including at least one convection tube extending through the endoscope, the at least one convection tube disposed in communication with the pressurized and regulated gas and/or air source, the at least one convection tube positioned adjacent to the at least one thermal component, the at least one convection tube configured to enable the insufflation gas to be pumped through the endoscope by the pressurized and regulated gas and/or air source for cooling the at least one thermal component by convection and for facilitating insufflation of the patient.

2. The robotic surgical system of claim 1, wherein the insufflation gas includes at least one of carbon dioxide, air, or an inert gas.

3. The robotic surgical system of claim 2, wherein the endoscope extends to an end effector on a distal end portion of the endoscope.

4. The robotic surgical system of claim 3, wherein the end effector defines at least one cooling passage therethrough that enables the insufflation gas to flow into or out of a distal end of the endoscope.

5. The robotic surgical system of claim 4, wherein the at least one cooling passage is disposed in communication with the at least one convection tube.

6. The robotic surgical system of claim 5, wherein the distal end portion further includes at least one temperature sensor configured to determine a temperature of at least one of the at least one thermal component or the at least one convection tube.

7. The robotic surgical system of claim 6, further comprising a controller, wherein the temperature sensor is disposed in communication with the controller, and wherein the pressurized and regulated gas and/or air source is connected to the controller whereby the controller selectively actives the pressurized and regulated gas and/or air source when the temperature sensor exceeds a predetermined threshold temperature.

8. The robotic surgical system of claim 7, further comprising a valve assembly coupled to the endoscope, the at least one convection tube disposed in communication with the valve assembly.

9. The robotic surgical system of claim 8, wherein the controller is configured to selectively actuate the valve assembly to adjust an amount of the insufflation gas being pumped through the at least one convection tube.

10. The robotic surgical system of claim 9, wherein the pressurized and regulated gas and/or air source is configured to apply positive pressure to the insufflation gas through the at least one convection tube, and further comprising a negative pressurized and regulated gas and/or air source operatively coupled to the endoscope, the negative pressurized and regulated gas and/or air source disposed in communication with the at least one convection tube and configured to apply negative pressure to the insufflation gas through the at least one convection tube.

11. A surgical system, comprising:
a pressurized and regulated gas and/or air source for pumping insufflation gas into a patient; and
an endoscope coupled to the pressurized and regulated gas and/or air source, the endoscope including at least one thermal component, the endoscope further including at least one convection tube extending through the endoscope, the at least one convection tube disposed in communication with the pressurized and regulated gas and/or air source, the at least one convection tube positioned adjacent to the at least one thermal component, the at least one convection tube configured to enable the insufflation gas to be pumped through the endoscope by the pressurized and regulated gas and/or air source.

12. The surgical system of claim 11, wherein the insufflation gas includes at least one of carbon dioxide, air, or an inert gas.

13. The surgical system of claim 12, wherein the endoscope extends to an end effector on a distal end portion of the endoscope, wherein the end effector defines at least one cooling passage therethrough that enables the insufflation gas to flow into or out of a distal end of the endoscope.

14. The surgical system of claim 13, wherein the at least one cooling passage is disposed in communication with the at least one convection tube.

15. The surgical system of claim 14, wherein the distal end portion further includes at least one temperature sensor configured to determine a temperature of at least one of the at least one thermal component or the at least one convection tube.

16. The surgical system of claim 15, further comprising a controller, wherein the temperature sensor is disposed in communication with the controller, and wherein the pressurized and regulated gas and/or air source is connected to the controller whereby the controller selectively actives the pressurized and regulated gas and/or air source when the temperature sensor exceeds a predetermined threshold temperature.

17. The surgical system of claim 16, further comprising a valve assembly coupled to the endoscope, the at least one convection tube disposed in communication with the valve assembly.

18. The surgical system of claim 17, wherein the controller is configured to selectively actuate the valve assembly to adjust an amount of the insufflation gas being pumped through the at least one convection tube.

19. The surgical system of claim 18, wherein the pressurized and regulated gas and/or air source is configured to apply positive pressure to the insufflation gas through the at least one convection tube, and further comprising a negative pressurized and regulated gas and/or air source operatively coupled to the endoscope, the negative pressurized and regulated gas and/or air source disposed in communication with the at least one convection tube and configured to apply negative pressure to the insufflation gas through the at least one convection tube.

20. A robotic surgical system, comprising:
a pressurized and regulated gas and/or air source for pumping insufflation gas into a patient;
an endoscope coupled to the pressurized and regulated gas and/or air source, the endoscope including at least one thermal component that is configured to generate heat and at least one temperature sensor configured to sense an amount of heat generated by the at least one thermal component, the endoscope further including at least one convection tube extending through the endoscope, the at least one convection tube disposed in communication with the pressurized and regulated gas and/or air source for receiving the insufflation gas through the endoscope;
a processor; and
a memory having instructions stored thereon, which when executed by the processor, cause the robotic surgical system to:
determine an amount of heat generated by the at least one thermal component; and
pump a predetermined amount of the insufflation gas through the at least one convection tube to cool the at least one thermal component by convection when the at least one temperature sensor senses that a temperature of the at least one thermal component exceeds a predetermined threshold.
